SEQUENCE LISTING (18 sequences)

SEQ ID NO: 1 - DNA, 717 bp, Artificial sequence
  oligonucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 2 - protein, 239 aa, Artificial sequence
  polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 3 - DNA, 675 bp, Artificial sequence
  oligonucleotide
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcc

SEQ ID NO: 4 - protein, 225 aa, Artificial sequence
  polypeptide
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 5 - DNA, 30 bp, Artificial sequence
  misc_feature: Primer
acaaagcttaatggtgagcaagggcgagga

SEQ ID NO: 6 - DNA, 32 bp, Artificial sequence
  misc_feature: Primer
tggacgagctgtacaagggatctgactacaag

SEQ ID NO: 7 - DNA, 66 bp, Artificial sequence
  misc_feature: Primer
gggtctgactacaaggacgatgacgataagggcggagactacaaggacgatgacgataagggctct

SEQ ID NO: 8 - DNA, 38 bp, Artificial sequence
  misc_feature: Primer
acgatgacgataagggctctatgcagatctttgtgagg

SEQ ID NO: 9 - DNA, 100 bp, Artificial sequence
  misc_feature: Primer
atgcagatctttgtgaggaccctcactggcagaaccatcacccttgaggtcgagcccagtgacaccattgagaatgtcagagccagaattcaagacaggg

SEQ ID NO: 10 - DNA, 99 bp, Artificial sequence
  misc_feature: Primer
gccagaattcaagacagggagggtatcccacctgaccagcagcgtctgatatttgccggcagacagctggaggatggccgcactctctcagactacaac

SEQ ID NO: 11 - DNA, 87 bp, Artificial sequence
  misc_feature: Primer
cactctctcagactacaacatccagagagagtccaccctgcacctggtgttgcgcctccgcggtggtatggcctcctccgaggacgt

SEQ ID NO: 12 - DNA, 20 bp, Artificial sequence
  misc_feature: Primer
atggcctcctccgaggacgt

SEQ ID NO: 13 - DNA, 40 bp, Artificial sequence
  misc_feature: Primer
ccgccactccaccggcgccggatctgactacaaggacgat

SEQ ID NO: 14 - DNA, 35 bp, Artificial sequence
  misc_feature: Primer
acaaggacgatgacgataagtagtaactcgagtca

SEQ ID NO: 15 - DNA, 22 bp, Artificial sequence
  misc_feature: Primer
cctggagacgccatccacgctg

SEQ ID NO: 16 - DNA, 49 bp, Artificial sequence
  misc_feature: Primer
taatacgactcactatagggagctcctcgcccttgctcaccattaagct

SEQ ID NO: 17 - DNA, 228 bp, Artificial sequence
  oligonucleotide
atgcagatctttgtgaggaccctcactggcagaaccatcacccttgaggtcgagcccagtgacaccattgagaatgtcagagccagaattcaagacagggagggtatcccacctgaccagcagcgtctgatatttgccggcagacagctggaggatggccgcactctctcagactacaacatccagagagagtccaccctgcacctggtgttgcgcctccgcggtggt

SEQ ID NO: 18 - protein, 76 aa, Artificial sequence
  polypeptide
MQIFVRTLTGRTITLEVEPSDTIENVRARIQDREGIPPDQQRLIFAGRQLEDGRTLSDYNIQRESTLHLVLRLRGG

(19) United States
(12) Patent Application Publication
(10) Pub. No.: US 2016/0068861 A1
(43) Pub. Date: Mar. 10, 2016

(54) METHOD FOR DETECTING PROTEIN STABILITY AND USES THEREOF

(71) Applicant: SHANGHAI INSTITUTES FOR BIOLIGICAL SCIENCES, CHINESE ACADEMY OF SCIENCES, Shanghai (CN)

(72) Inventors: Ronggui HU, Shanghai (CN); Tao YU, Shanghai (CN); Yonghui TAO, Shanghai (CN)

(21) Appl. No.: 14/783,906

(22) PCT Filed: Apr. 11, 2014
(86) PCT No.: PCT/CN2014/075216

§ 371 Date: Oct. 12, 2015

(30) Foreign Application Priority Data
Apr. 12, 2013 (CN) 201310128492.8

(51) Int. Cl. C12N 15/85; C07K 14/435; C12N 15/62

(52) U.S. Cl. CPC C12N 15/85; C12N 15/62; C07K 14/435; C07K 2319/00; C07K 2319/60

(57) ABSTRACT

Provided in the present invention is a genetic construct having a structure as represented by 5′-A+B+C+D+E-3′, wherein A indicates a promoter; B indicates a coding sequence of a fusion protein consisting of a target protein and a first labeled protein; C indicates a coding sequence of a connecting peptide; D indicates a coding sequence of a second labeled protein; and E indicates a terminator. Also provided in the present invention is a polypeptide having a structure as represented by B1+C1+D1, wherein B1 indicates the fusion protein consisting of the target protein and the first labelled protein; C1 indicates the connecting peptide; and D1 indicates a second labelled protein. Also provided in the present invention are a vector containing the genetic construct, a mammalian cell containing the genetic construct or the vector, a library consisting of the cell and a method for detecting protein stability and uses thereof. The method of the present invention not only has a high sensitivity and specificity for detecting protein stability, but also is simple and convenient in operation.

### TECHNICAL FIELD

The present invention relates to the field of detection of proteins, and in particular, to a method for detecting protein stability and uses thereof.

### BACKGROUND

Protein dynamics in protein level are essential features and prerequisites for a cell to maintain viability and perform life activities. At any time, the level of a specific protein in a cell depends on the dynamic equilibrium between synthesis and degradation of the protein. Proteins are mainly synthesized on ribosome, and at present, processes mechanisms for the synthesis and regulation of a protein are well understood.

Research on protein degradation in human began in 1980's, and in recent years, has become a very important area on modern biology, because basic life processes of a cell, such as cell cycle, are not possible, unless all of the proteins are timely degraded. Numerous evidences showed that many damaged or misfolded proteins must be degraded; otherwise normal cell function will be affected, resulting in diseases such as cancer, neurodegenerative diseases (C. Raiborg and H. Stenmark, 2009; Christian Hirsch et, 2009; Steven Bergink and Stefan Jentsch, 2009).

Abnormal protein degradation pathways are involved in many pathological processes (Salvatore Oddo, 2008; Jeffrey H Kordower, 2008; Ji anjun Wang, 2008), therefore, many tumor cells and viruses also take advantage of the ubiquitin-proteasome system to disrupt protein degradation, so as to achieve the distribution of tumor cells or their invasion into host cells (Daniela Hoeller and Ivan Dikic, 2009). Many drugs which can affect protein degradation pathways have been or are being developed. Existing research results and data from clinical trials have demonstrated that these drugs have high potential medicinal value for many currently incurable diseases (Matthew D Petroski, 2008; Grzegorz Nalepa, 2006).

Therefore, studies on the change in protein stability will provide important insights on the forecast, incidence, etiology of the protein-involved diseases, and the screening of therapeutic agents. Particularly, the detection of abnormal stability of a protein is very important for the study of pathogenesis. At present, the stability of a protein is detected through cycloheximide chase assay. However, this method is labor-consuming, and the changes in protein stability within the scope of the proteomics can not be studied. And such assay is not suitable for high-throughput drug screening due to its complexity.

By ProteinChip technology, the stabilities of multiple proteins can be detected in one time. However, the throughput of the technology is limited due to the availability of antibodies, thereby greatly limiting the practicability of the technology. Changes in protein stability within the scope of proteomics can be detected by mass spectrometry in large-scale samples. However, mass spectrometry has limited capability of identification, and changes in stability of low-abundance protein can not be detected.

In the field of cell biology and molecular biology, the fluorescent proteins are widely used. Fluorescent proteins are often used as reporter genes to identify the cellular localization of a target protein, and can be used in combination with FACS technology for relatively quantitating intracellular abundance of a target protein.

Therefore, there is an urgent need in the art to develop a novel detection method, which can be widely used for genome-wide detection of the stability of a protein, and readily applied to high-throughput screen of small compounds.

### SUMMARY OF INVENTION

The aim of the present invention is to provide a fusion protein for detecting protein stability and a genetic construct encoding the fusion protein, a vector comprising said genetic construct, a cell comprising said genetic construct or vector, and a library consisting of said cell, and a method for detecting protein stability and uses thereof. The method of the present invention for detecting protein stability is of high sensitivity and specificity, and easy to operate.

In the first aspect, the present invention provides a genetic construct as follows:

5′-A+B+C+D+E-3′,

wherein,

A represents a promoter;

B represents a coding sequence for a fusion protein consisting of a target protein and a first marker protein;

C represents a coding sequence for a linker peptide;

D represents a coding sequence for a second marker protein; and

E represents a terminator;

wherein B and D can be interchanged.

In a preferred embodiment, the promoter is a CMV promoter.

In another preferred embodiment, B, from 5′ to 3′, comprises a coding sequence for the target protein and a coding sequence for the first marker protein.

In another preferred embodiment, the linker peptide is selected from: ubiquitin, truncated ubiquitin, ubiquitin mutants and ubiquitin-like proteins, 2A peptides and the like; preferably, ubiquitin; most preferably, ubiquitin with saturation K(R) mutation.

In a preferred embodiment, the first marker protein and the second marker protein are fluorescent proteins.

In a preferred embodiment, the fluorescent protein is selected from EGFP or RFP; and preferably, the first marker protein is EGFP, and the second marker protein is RFP.

In a preferred embodiment, EGFP is mEGFP, and RFP is mRFP.

In a preferred embodiment, one or more tags, such as Flag or myc, are fused at C-terminal or N-terminal of the fluorescent protein.

In another preferred embodiment, the coding sequence of the target protein comprises all of the genes in human genomic library human ORFeome V5.1.

In a second aspect, the present invention provides a polypeptide with a structure of the following formula:

B1+C1+D1,

wherein,

B1 represents a fusion protein consisting of a target protein and a first marker protein;

C1 represents a linker peptide; and

D1 represents a second marker protein;

wherein B1 and D1 can be interchanged.

In a preferred embodiment, each part in the polypeptide successively is the target protein, the first marker protein, linker peptide and the second marker protein, or the second marker protein, the linker peptide, the target protein and the first marker protein.

In a preferred embodiment, the polypeptide is encoded by genetic construct of claim 1. In another preferred embodiment, the linker peptide is selected from: ubiquitin, ubiquitin mutants and ubiquitin like proteins, 2A peptides and the like; preferably, ubiquitin; most preferably, ubiquitin with saturation K(R) mutation.

In another preferred embodiment, the first marker protein and the second marker protein are fluorescent proteins.

In a preferred embodiment, the fluorescent protein is selected from EGFP or RFP; and preferably, the first marker protein is EGFP, and the second marker protein is RFP.

In another preferred embodiment, EGFP is mEGFP, and RFP is mRFP.

In another preferred embodiment, one or more tags, such as Flag or myc, are fused at C-terminal or N-terminal of the fluorescent protein.

In another preferred embodiment, the target protein comprises all of the proteins encoded by open reading frames in human genomic library human ORFeome V5.1.

In the third aspect, the present invention provides a vector comprising the genetic construct according to the first aspect of the present invention.

In a preferred embodiment, the vector can be a plasmid, such as a retroviral plasmid, a lentivirus plasmid, which can be integrated into cell genome of a recipient.

In the fourth aspect, the present invention provides a cell comprising the genetic construct according to the first aspect of the present invention, or the vector according to the third aspect of the present invention.

In the fifth aspect, the present invention provides a cell library consisting of the cells of the fourth aspect of the present invention.

In a preferred embodiment, said cell is a mammalian cell;

In another preferred embodiment, the mammalian cell is a primate cell or a human cell; and preferably, the mammalian cell is a human cell.

In another preferred embodiment, the mammalian cells include, but are not limited to: 293 cells, 293T cells, 293FT cells, Hela cells, NIH3T3 cells, cancer cells, stem cells, and the like.

In the sixth aspect, the present invention provides a method for detecting the stability of one or more target proteins, comprising the following steps:

(1) constructing the cell library according to the fifth aspect of the present invention;

(2) culturing the cell library obtained in (1) under the specific conditions and control conditions;

(3) determining the ratio of the first marker protein to the second marker protein in the cell library cultured under the specific conditions and control conditions; and

(4) drawing a conclusion that the stability of the target protein is changed or not, based on the ratio of the first marker protein to the second marker protein in the cell library cultured under the specific conditions and control conditions; or:

separating the cell into n sections (n≧2) through flow cytometry based on the ratio of the first marker protein to the second marker protein, detecting the expression of each target gene in each section by DNA chip technology to calculate the distribution of the target genes in each section, and comparing the distributions of target genes in cell libraries cultured under the specific conditions and control conditions, thereby drawing a conclusion that the stability of the protein encoded by the target gene is changed or not.

In a preferred embodiment, the specific condition can be the addition of a stimulus and disturbance, for example, a chemical stimulus, such as a drug; gene overexpression, knockout or RNA interference; viral infection, stable transfection, plasmid transient transfection, or can be a control condition itself.

In a preferred embodiment, the specific condition refers to the contact with a compound to be tested, stress, radiation or other conditions; and preferably, the specific condition refers to the contact with a compound to be tested.

In a preferred embodiment, the ratio of the first marker protein to the second marker protein, i.e., the ratio of fluorescence intensities of the fluorescent proteins is detected by flow cytometry.

In a preferred embodiment, the number of the sections is 8.

In a preferred embodiment, the target protein comprises all of the proteins encoded by all of ORF in human genomic library human ORFeome V5.1.

It should be understood that in the present invention, the technical features specifically mentioned above and below (such as in the Examples) can be combined with each other, thereby constituting a new or preferred technical solution which needs not be individually described.

### DESCRIPTION OF THE DRAWINGS

FIG. 1a is a schematic diagram of the construct of a dual-fluorescent plasmid.

FIG. 1b is the principle of detecting the relative stability of a protein by using dual-fluorescent plasmid system.

FIG. 1c shows that mEGFP without fusion of protein ORF is stable, and forms a stable molecule with mRFP at a molar ratio of 1:1.

FIG. 1d shows that RGS4, the substrate protein of anticancer drugs Bortezomib, was successfully screened using dual-fluorescence plasmid system, and there is no change for negative protein. Western data validation is attached.

FIG. 2a is a schematic diagram of genome-wide determination of the stability of a protein by using dual-fluorescence cell library.

FIG. 2b is PSI data distribution of genome-wide determination of the relative stability of a protein by using dual-fluorescence cell library.

FIGS. 2c and d show PSI data of randomly selected genes.

FIG. 3a shows the chemical structure of anticancer drug Bortezomib.

FIG. 3b shows the overall changes of whole dual-fluorescence cell library upon treatment of anticancer drug Bortezomib.

FIG. 3c shows representative data.

FIG. 3d is Δ PSI distribution representing the changes in protein relative stability.

FIGS. 3e, f, g and h show data validation of chip results using FACS and Western Blot.

FIG. 4a shows GO (Biological Pathway) analysis on proteins with dramatic changes in stability using bioinformatics tool DAVID.

FIG. 4b shows the detail analysis performed on BP004 subfamily in the results of FIG. 4a.

FIG. 4c shows the detail analysis performed on BP00179 subfamily in the results of FIG. 4a.

FIG. 4d shows protein-protein interactions (PPI) analysis performed on response proteins by using bioinformatics tool STRING and visualization tool cytoscape.

FIG. 5 shows scatter plots of the control dual-fluorescent cell line.

FIG. 6 shows the dual-fluorescent cell library containing human ORFeome V5.1.

FIG. 7, using TP53-mEGFPfu-mRFPf as an example, shows the results of western blot of original band of the fusion polypeptide which has just been translated, and two different bands (TP53-mEGFP fusion protein band and mRFP band) formed through cleavage by deubiquitinating enzyme.

FIG. 8 shows small compound-protein interactions (CPI) analysis performed on response proteins by using bioinformatics tool STITCH and visualization tool cytoscape.

FIGS. 9a and b show the results from using BTZ in combination with 17-AAG against CZ-1 or CZ-1/R cells.

FIGS. 9c and d show the results from using BTZ in combination with C75 against CZ-1 or CZ-1/R cells.

FIG. 10a shows CI calculation results for the combination of BTZ with C75.

FIG. 10b shows CI calculation results for the combination of BTZ with 17-AAG.

### DETAILED DESCRIPTION

Through comprehensive and intensive research, the inventors have unexpectedly found that a dual-fluorescence plasmid system of the present invention can be used to achieve an easy and accurate genome-wide detection of the protein stability, thereby significantly improving the practicability of dual fluorescence technology, and such technology can be further applied to high-throughput screening of small compounds affecting the stability of proteins of pathogenic genes and find unknown key genes involved in pathogenesis of diseases. Based on the above findings, the present invention is completed.

In the present invention, the inventors have designed a dual-fluorescent plasmid system for relative quantification of the protein degradation rate. The key point of the technology is co-expression of a fluorescent reporter protein molecule (mEGFP) which is fused to a specific target protein, and a control fluorescent protein molecule (mRFP), both of which are connected by a specific linker protein, and upon the translation, will be effectively cleaved and separated. The expression level of mRFP serves as an internal reference, and mEGFP is fused to C-terminus of the target protein X, and serve as a measure for intracellular abundance of target protein X. Through mEGFP/mRFP ratio, the inventor can accurately define the stability of the specific protein X in a single cell.

And if any biological event (such as deletion or overexpression of E3 ligase for adjusting the specific protein X) or chemical stimulus (such as drug treatment) selectively alters the stability of the fusion protein X-mEGFP, the abundance of the fusion protein X-mEGFP will be changed, however, the abundance of mRFP won't be affected, thereby changing the mEGFP/mRFP ratio.

Accordingly, the present invention can effectively overcome the disadvantages in the prior art and widely applied to the genome-wide determination of the stability of a protein and high-throughput screening of small compounds.

In the present invention, a fusion protein consisting of a target protein, a first marker protein, linker peptide and a second marker protein is co-expressed under the same promoter, and a fusion protein of the target protein and the first marker protein and the second protein form respectively upon cutting at the linker peptide, thereby characterizing the relative stability of the target protein by comparison between the first marker protein fused to the target protein and the second marker protein.

For example, in a specific embodiment, according to the present invention, a fluorescent protein is fused to a target gene or a gene library, and the amount of the fusion protein can be characterized based on fluorescent intensity since fluorescent intensity of a fluorescent molecule can be quantitatively determined, so as to obtain parameters regarding protein stability. The simultaneous genome-wide detection of stability of all proteins can be achieved by combining with human ORFeome library genes.

Using this technique, small compounds which can affect the stability of proteins of pathogenic genes can be selected through high-throughput screening. Meanwhile, by combining with human ORFemoe library, genome-wide detection of the stability of a protein can be achieved, and a protein, the stability of which changes between disease state and normal state, can be genome-widely screened, so as to find unknown key genes involved in pathogenesis of diseases for understanding diseases which can not be thoroughly understood yet.

In a preferred embodiment, the dual-fluorescent expression plasmid according to the present invention is constructed as follows:

a control protein ((Red fluorescent protein (RFP)) and a specific target protein fused to a fluorescent reporter protein (enhanced green fluorescent protein (EGFP)) are co-expressed under the same promoter, both of which are connected by ubiquitin, and upon translation, ubiquitin will be effectively cleaved by hundreds of deubiquitinating enzymes in a cell so as to form separated RFP. The expression level of RFP serves as an internal reference, and EGFP is fused to C-terminus of the target protein X and become a measure means for intracellular abundance of target protein X. Through EGFP/RFP ratio, the relative stability of the specific protein X in a single cell can be accurately defined, and cells with different EGFP/RFP ratios can be sorted and detected by flow cytometry. When performing high-throughput screening of drugs, the high-throughput screening can be conducted by linking a target protein into the dual-fluorescent expression vector through efficient homologous recombination technology Gateway reaction, and then constructing a cell line. When it is desired to genome-widely detect abnormal changes in the stability of a protein, all of the genes in human ORFemoe library are recombinantly linked into the dual-fluorescent expression plasmid through homologous recombination technology Gateway reaction, and then cells are infected at ultra-low viral titers (MOI ˜0.05), a cell library into which human ORFemoe library is integrated is constructed at a frequency of one-gene per one-cell, and genome-wide detection of the stability of all the proteins can be conducted in one time by using such cell library in combination of FACS and chip technology.

Accordingly, the present invention provides a genetic construct with a structure shown in the following formula:

5′-A+B+C+D+E-3′,

wherein,

A represents a promoter; B represents a coding sequence for a fusion protein consisting of a target protein and a first marker protein; C represents a coding sequence for a linker peptide; D represents a coding sequence for a second marker protein; and E represents a terminator; wherein B and D can be interchanged.

Wherein, the order of the coding sequence for the target protein and the first marker protein can be interchanged.

In light of the teachings of the present invention and the prior art, a person skilled in the art will appreciate that, in the present invention, a fusion protein consisting of a target protein, a first marker protein, linker peptide and a second marker protein is co-expressed, a fusion protein of the target protein and the first marker protein and the second protein form respectively upon cleaving at the linker peptide, thereby characterizing the relative stability of the target protein by comparison between the first marker protein fused to the target protein and the second marker protein. Therefore, B and D in the above genetic construct can be interchanged without affecting the function or use thereof.

In light of the teachings of the present invention and the prior art, a person skilled in the art will further appreciate that the order of the coding sequence for the target protein and the coding sequence for the first marker protein in B can be interchanged without affecting the function or use thereof

In a preferred embodiment, B, from 5′ to 3′, comprises the coding sequence for the target protein and the coding sequence for the first marker protein, respectively.

In light of the teachings of the present invention and the prior art, a person skilled in the art will appreciate that various of linker peptide can be used in the present invention, as long as the linker peptide can be intracellularly cleaved to form two separate marker proteins. For example, in a particular embodiment, the linker peptide is selected from: ubiquitin (Ub), ubiquitin mutants and ubiquitin-like proteins, 2A short peptides and the like; preferably ubiquitin; most preferably, ubiquitin with K(R) mutation. Ubiquitin with K(R) mutation can be used to ensure effective cleavage, while won't become a ubiquitination site, and thus the degradation properties of the target protein will not be affected.

A person skilled in the art will further appreciate that various marker proteins can be used in the present invention, as long as the amount of the marker protein can be accurately characterized. For example, in a particular embodiment, the first and the second marker protein are fluorescent proteins; preferably, the fluorescent protein is EGFP or RFP; and more preferably, the first marker protein is EGFP, and the second marker protein is RFP. In a more preferred embodiment, EGFP is EGFP in monomer form (mEGFP), and RFP is RFP in monomer form (mRFP).

In another preferred embodiment, one or more label proteins, such as Flag or myc, are fused at C-terminal or N-terminal of the fluorescent protein.

In another preferred embodiment, the promoter is a CMV promoter.

In another preferred embodiment, the coding sequences of the target proteins are all of the genes in human genomic library.

In the present invention, a polypeptide with a structure of the following formula is further provided:

B1+C1+D1,

wherein,

B1 represents a fusion protein consisting of a target protein and a first marker protein;

C1 represents a linker peptide; and

D1 represents a second marker protein;

wherein B1 and D1 can be interchanged.

As said above, a person skilled in the art will appreciate that B1 and D1 in the polypeptide of the present invention can be interchanged without affecting the function or use thereof. And the order of the target protein and the first marker protein in the polypeptide of the present invention can be interchanged without affecting the function or use thereof.

In a preferred embodiment, the polypeptide is encoded by the genetic construct of the present invention.

In a preferred embodiment, the order of each part in the polypeptide is the target protein, the first marker protein, the linker peptide and the second marker protein, or the second marker protein, the linker peptide, the target protein and the first marker protein.

Based on the genetic construct of the present invention, a vector comprising the genetic construct of the present invention, a mammalian cell comprising the genetic construct or the vector, and a mammalian cell library consisting of the mammalian cells are also provided in the present invention.

In a specific embodiment, the vector can be a plasmid, such as a retroviral plasmid, a lentivirus plasmid, which can be integrated into cell genome of a recipient. Said mammalian cell can be a primate cell or a human cell; and preferably, said mammalian cell is a human cell. In a specific embodiment, the mammalian cell includes, but not limited to: 293 cells, and can be other cells, such as 293t, hela, 3t3, cancer cell, stem cell, and the like.

Based on the genetic construct, vector, polypeptide, cell and cell library of the present invention, a method for detecting the change in the stability of a target protein under specific conditions, comprising the following steps:

(1) constructing the cell library according to the present invention;

(2) culturing the obtained cell library under the specific conditions and control conditions;

(3) determining the ratio of the first marker protein to the second marker protein in the cell library cultured under the specific conditions and control conditions; and

(4) drawing a conclusion that the stability of the target protein is changed or not, based on the ratio of the first marker protein to the second marker protein in the cell library cultured under the specific conditions and control conditions.

After the cell library is obtained, the addition of a stimulus and disturbance can be applied, for example, a chemical stimulus, such as a drug; gene overexpression or RNA interference; viral stable transfection, or plasmid instantaneous transfection.

In a preferred embodiment, the specific condition refers to the contact with a compound to be tested, stress, radiation and other conditions; and preferably, the specific condition refers to the contact with a compound to be tested.

In a preferred embodiment, DNA microarrays are used to detect the ratio of the first marker to the second marker protein, for example, to detect the ratio of fluorescence intensity of the first marker to that of the second marker protein.

In a preferred embodiment, the target proteins are all of the proteins in human cell genomic library human ORFeome V5.1.

In the present invention, the involved genome-wide gene can be human ORF version 5.1, or higher version or other versions, or can be ORF library versions of other species.

In light of the teachings of the present invention and the prior art, a person skilled in the art will appreciate that a variety of marker proteins can be used in the present invention, so long as the amount of the marker proteins can be accurately characterized. For example, a variety of fluorescent proteins can be used in the present invention, including but not limited to mEGFP and mRFP or a mutant thereof; alternatively, other fluorescent proteins can be used, as long as two fluorescent proteins possess different excitation light wavelength.

Two different fluorescent proteins are used in the polypeptides of the present invention. In a preferred embodiment, the fluorescent proteins used in the present invention are mEGFP and mRFP.

For example, the fluorescent protein of SEQ ID NO: 1, 2, 3 or 4 can be used in the present invention (SEQ ID NO: 1, atggtgagcaagggcgaggagctgttcaccggggtggtgcccatcctggtcgagctggacggcgacgtaa acggccacaagttcagcgtgtccggcgagggcgagggcgatgccacctacggcaagctgaccctgaagttcatctgcaccacc ggcaagctgcccgtgccctggcccaccctcgtgaccaccctgacctacggcgtgcagtgcttcagccgctaccccgaccacatg aagcagcacgacttcttcaagtccgccatgcccgaaggctacgtccaggagcgcaccatcttcttcaaggacgacggcaactaca agacccgcgccgaggtgaagttcgagggcgacaccctggtgaaccgcatcgagctgaagggcatcgacttcaaggaggacgg caacatcctggggcacaagctggagtacaactacaacagccacaacgtctatatcatggccgacaagcagaagaacggcatcaa ggtgaacttcaagatccgccacaacatcgaggacggcagcgtgcagctcgccgaccactaccagcagaacacccccatcggcg acggccccgtgctgctgcccgacaaccactacctgagcacccagtccgccctgagcaaagaccccaacgagaagcgcgatcac atggtcctgctggagttcgtgaccgccgccgggatcactctcggcatggacgagctgtacaag; SEQ ID NO: 2, MVSKGEELFTGVVPILVELDGDVNGHKFSVSGEGEGDATYGKLTLKFICTTGKLPV PWPTLVTTLTYGVQCFSRYPDHMKQHDFFKSAMPEGYVQERTIFFKDDGNYKTRA EVKFEGDTLVNRIELKGIDFKEDGNILGHKLEYNYNSHNVYIMADKQKNGIKVNF KIRHNIEDGSVQLADHYQQNTPIGDGPVLLPDNHYLSTQSK(A)LSKDPNEKRDHM VLLEFVTAAGITLGMDELYK; SEQ ID NO: 3, atggcctcctccgaggacgtcatcaaggagttcatgcgc ttcaaggtgcgcatggagggctccgtgaacggccacgagttcgagatcgagggcgagggcgagggccgcccctacgagggca cccagaccgccaagctgaaggtgaccaagggcggccccctgcccttcgcctgggacatcctgtcccctcagttccagtacggctc caaggcctacgtgaagcaccccgccgacatccccgactacttgaagctgtccttccccgagggcttcaagtgggagcgcgtgatg aacttcgaggacggcggcgtggtgaccgtgacccaggactcctccctgcaggacggcgagttcatctacaaggtgaagctgcgc ggcaccaacttcccctccgacggccccgtaatgcagaagaagaccatgggctgggaggcctccaccgagcggatgtaccccga ggacggcgccctgaagggcgagatcaagatgaggctgaagctgaaggacggcggccactacgacgccgaggtcaagaccac ctacatggccaagaagcccgtgcagctgcccggcgcctacaagaccgacatcaagctggacatcacctcccacaacgaggacta caccatcgtggaacagtacgagcgcgccgagggccgccactccaccggcgcc; SEQ ID NO: 4, MASSEDVIKE FMRFKVRMEGSVNGHEFEIEGEGEGRPYEGTQTAKLKVTKGGPLPFAWDILSPQF QYGSKAYVKHPADIPDYLKLSFPEGFKWERVMNFEDGGVVTVTQDSSLQDGEFIY KVKLRGTNFPSDGPVMQKKTMGWEASTERMYPEDGALKGEIKMRLKLKDGGHY DAEVKTTYMAKKPVQLPGAYKTDIKLDITSHNEDYTIVEQYERAEGRHSTGA).

The present invention includes a fluorescent protein comprising the amino sequence of SEQ ID NO: 2 or 4, and mutants thereof having the same function as SEQ ID NO: 2 or 4, or other fluorescent proteins. Possible fluorescent proteins are listed in Table 1, including but not limited to the following proteins:

TABLE 1

| | | | EC/10−3 | | | Relative |
|---|---|---|---|---|---|---|
| | | | M−1 | | quarternary | brightness |
| Protein (acronym) | Ex/nm | Em/nm | cm−1 | QY | structure | (% EGFP) |
| Blue fluorescent protein | | | | | | |
| Sirius | 355 | 424 | 15 | 0.24 | monomer* | 11 |
| Azurite | 384 | 448 | 26.2 | 0.55 | monomer* | 43 |
| EBFP | 383 | 445 | 29 | 0.31 | monomer* | 27 |
| EBFP2 | 383 | 448 | 32 | 0.56 | monomer* | 53 |
| Cyan fluorescent protein | | | | | | |
| ECFP | 439 | 476 | 32.5 | 0.4 | monomer* | 39 |
| Cerulean | 433 | 475 | 43 | 0.62 | monomer* | 79 |
| CyPet | 435 | 477 | 35 | 0.51 | monomer* | 53 |
| SCFP | 433 | 474 | 30 | 0.5 | monomer* | 45 |
| Green fluorescent protein | | | | | | |
| EGFP | 488 | 507 | 56 | 0.6 | monomer* | 100 |
| Emerald | 487 | 509 | 57.5 | 0.68 | monomer* | 116 |
| Superfolder avGFP | 485 | 510 | 83.3 | 0.65 | monomer* | 160 |
| T-Sapphire | 399 | 511 | 44 | 0.6 | monomer* | 79 |
| Yellow fluorescent protein | | | | | | |
| EYFP | 514 | 527 | 83.4 | 0.61 | monomer* | 151 |
| Topaz | 514 | 527 | 94.5 | 0.6 | monomer* | 169 |
| Venus | 515 | 528 | 92.2 | 0.57 | monomer* | 156 |
| Citrine | 516 | 529 | 77 | 0.76 | monomer* | 174 |
| YPet | 517 | 530 | 104 | 0.77 | monomer* | 238 |
| SYFP | 515 | 527 | 101 | 0.68 | monomer* | 204 |
| mAmetrine | 406 | 526 | 45 | 0.58 | monomer | 78 |
| Blue fluorescent protein | | | | | | |
| mTagBFP | 399 | 456 | 52 | 0.63 | monomer | 98 |
| Cyan fluorescent protein | | | | | | |
| TagCFP | 458 | 480 | 37 | 0.57 | monomer | 63 |
| AmCyan | 458 | 489 | 44 | 0.24 | tetramer | 31 |
| Midoriishi Cyan | 472 | 495 | 27.3 | 0.9 | dimmer | 73 |
| mTFP1 | 462 | 492 | 64 | 0.85 | monomer | 162 |
| Green fluorescent protein | | | | | | |
| Azami Green | 492 | 505 | 55 | 0.74 | monomer | 121 |
| mWasabi | 493 | 509 | 70 | 0.8 | monomer | 167 |
| ZsGreen | 493 | 505 | 43 | 0.91 | tetramer | 117 |
| TagGFP | 482 | 505 | 58.2 | 0.59 | monomer | 102 |
| TagGFP2 | 483 | 506 | 56.5 | 0.6 | monomer | 105 |
| TurboGFP | 482 | 502 | 70 | 0.53 | dimer | 112 |
| CopGFP | 482 | 502 | 70 | 0.6 | tetramer | 125 |
| AceGFP | 480 | 505 | 50 | 0.55 | monomer | 82 |
| Yellow fluorescent protein | | | | | | |
| TagYFP | 508 | 524 | 64 | 0.6 | monomer | 118 |
| TurboYFP | 525 | 538 | 105 | 0.53 | monomer | 169 |
| ZsYellow | 529 | 539 | 20.2 | 0.42 | tetramer | 25 |
| PhiYFP | 525 | 537 | 130 | 0.4 | dimmer | 158 |
| Orange fluorescent protein | | | | | | |
| Kusabira Orange | 548 | 559 | 51.6 | 0.6 | monomer | 92 |
| Kusabira Orange2 | 551 | 565 | 63.8 | 0.62 | monomer | 118 |
| mOrange | 548 | 562 | 71 | 0.69 | monomer | 146 |
| mOrange2 | 549 | 565 | 58 | 0.6 | monomer | 104 |
| dTomato | 554 | 581 | 69 | 0.69 | dimmer | 142 |
| dTomato-Tandem | 554 | 581 | 138 | 0.69 | pseudo-monomer | 283 |
| DsRed | 558 | 583 | 75 | 0.79 | tetramer | 176 |
| DsRed2 | 563 | 582 | 43.8 | 0.55 | tetramer | 72 |
| DsRed-Express (T1) | 555 | 584 | 38 | 0.51 | tetramer | 58 |
| DsRed-Express2 | 554 | 586 | 35.6 | 0.42 | tetramer | 45 |
| DsRed-Max | 560 | 589 | 48 | 0.41 | tetramer | 59 |
| DsRed-monomer | 556 | 586 | 35 | 0.1 | monomer | 10 |
| TurboRFP | 553 | 574 | 92 | 0.67 | dimmer | 187 |
| TagRFP | 555 | 584 | 100 | 0.48 | monomer | 142 |
| TagRFP-T | 555 | 584 | 81 | 0.41 | monomer | 99 |
| Red fluorescent protein | | | | | | |
| mRuby | 558 | 605 | 112 | 0.35 | monomer | 117 |
| mApple | 568 | 592 | 75 | 0.49 | monomer | 109 |
| mStrawberry | 574 | 596 | 90 | 0.29 | monomer | 78 |
| AsRed2 | 576 | 592 | 56.2 | 0.05 | tetramer | 8 |
| mRFP1 | 584 | 607 | 50 | 0.25 | monomer | 37 |
| JRed | 584 | 610 | 44 | 0.2 | dimer | 26 |
| mCherry | 587 | 610 | 72 | 0.22 | monomer | 47 |
| eqFP611 | 559 | 611 | 78 | 0.45 | tetramer | 106 |
| tdRFP611 | 558 | 609 | 70 | 0.47 | pseudo-monomer | 98 |
| HcRed1 | 588 | 618 | 20 | 0.015 | dimmer | 1 |
| mRaspberry | 598 | 625 | 86 | 0.15 | monomer | 38 |
| Far infrared fluorescent protein | | | | | | |
| tdRFP639 | 589 | 631 | 90.4 | 0.16 | pseudo-monomer | 43 |
| mKate | 588 | 635 | 31.5 | 0.28 | monomer | 26 |
| mKate2 | 588 | 633 | 62.5 | 0.4 | monomer | 74 |
| Katushka | 588 | 635 | 65 | 0.34 | dimer | 67 |
| tdKatushka | 588 | 633 | 132.5 | 0.37 | pseudo-monomer | 146 |
| HcRed-Tandem | 590 | 637 | 160 | 0.04 | pseudo-monomer | 19 |
| mPlum | 590 | 649 | 41 | 0.1 | monomer | 12 |
| AQ143 | 595 | 655 | 90 | 0.04 | tetramer | 11 |

Note:
In the table, the peak excitation wavelength is Ex, the peak emission wavelength is Em, molar extinction coefficient is EC, and quantum yield is QY. Brightness value is calculated from the product of molar extinction coefficient and quantum yield divided by EGFP.

For example, a fluorescent protein, such as mEGFP may be fused, at N terminal, to ORF of a target protein, that is, the used structure of the fusion protein is: ORF-mEGFP-Ub-mRFP; or, fused, at C terminal, to ORF of a target protein, that is, the used structure of the fusion protein is: mEGFP-ORF-Ub-mRFP; alternatively, for example, mRFP can be fused to ORF of a target protein.

The fluorescent molecule of the present invention may have 2 Flag labels fused at C-terminal, or not; alternatively, one or more Flag labels can be fused, or other labels, such as myc and the like, can be used. The label can also be fused at N-terminal of the fluorescent molecule.

In practice, based on the present disclosure in combination with the amino acid composition and structure of fluorescent proteins known in the art, a person skilled in the art can select the type of fluorescent protein molecules to be fused at N-terminal or C-terminal by himself based on the experimental requirement and purpose.

The present invention includes the use of ORF fused dual-fluorescent protein to detect the stability of a protein in various libraries. The libraries used in the present invention can be commercially-available ORF libraries, or libraries constructed by a person skilled in the art themselves, or human ORF, or ORFs of other species, or a part of function domains in ORF, or a sequence fragment designed by a person skilled in the art himself.

Recombination and amplification of genes in a library are performed in E. coli. Competent cells which can absorb DNA can be harvested after exponential growth phase, and treated with CaCl₂, the procedure of which is well known in the art. Another method is to use MgCl₂. If desired, transformation can be conducted by electroporation.

Once sequences of library genes for constructing a library are obtained, gene library of fused dual-fluorescent protein molecules can be constructed. The construction method can be gene recombination method, for example, Gateway reaction, or other recombination reactions. The method may also be conventional recombinant DNA techniques (Science, 1984; 224: 1431), for example conventional digestion-ligase linking Method.

Upon obtaining a gene library of fused dual-fluorescent protein molecules, dual-fluorescent cell library with integrated plasmid gene can be constructed through viral packaging methods. The plasmid can be lentivirus plasmid or other plasmids into which genome can be integrated, and the cell for constructing the dual-fluorescence cell library can be 293 cell, or can be other cells, such as 293T cell, Hela cell, NIH3T3 cell, cancer cell, stem cell and the like.

The constructed library can be sorted by FASC, and separated into several sections based on mEGFP/mRFP ratio, wherein the number of sections can be 6, 7, 8, 9 or more; alternatively, the cell in some sections with mEGFP/mRFP ratio in the library can be refine-sorted. The number of sections can be 4, 5, 6, 7 or more.

Genome from sorted cells can be extracted respectively through conventional extraction methods for extracting genomic DNA from a cell or tissue and preparing a sample containing genomic DNA. Primers are designed using common segments of plasmid and genomic DNAs as templates. ORFs are amplified through PCR, T7 promoter primer is added to 3′ end primer, and tag enzymes used in PCR amplification suitably are enzymes with a strong ability to extend, such as Ex-tag and the like. PCR products are in vitro RNA-amplified, amplified products are used in chip hybridization. Chip can be Agilent double standard chip. Results from chips are normalized by using Composite loess normalization in Bioconductor package limma.

The features of the present invention mentioned above, or the features mentioned in the examples, can be optionally combined. Any feature disclosed in the present specification can be used in combination with any other features, and each feature disclosed in the specification can be replaced with alternative feature which can serve an identical, equivalent, or similar purpose. Therefore, the features disclosed herein are only general exemplary examples of the equivalent or similar features, unless specifically indicated otherwise.

Main advantages of the present invention:

1. The practicability of detecting protein stability is greatly improved by the present invention;

2. The method of the present invention is of high detection accuracy, and convenient operation, and time-saving;

3. Changes in protein stability can be detected with high sensitivity and specificity by the method of the present invention;

4. In the method of the present invention, the fluorescent protein molecule is fused to C segment of the target protein, therefore, signal sequence at N terminal of a protein can be effectively exposed, thereby factually simulating dynamic degradation of a protein;

5. In the method of the present invention, ubiquitin Ub is used to link the fluorescent protein molecules, Ub can be high effectively cleaved by large number of intracellular deubiquitinating enzymes, thereby ensuring 1:1 molar ratio between two fluorescent molecules at the protein translation level;

6. In the method of the present invention, monomeric form of fluorescent molecule is used, which can effectively prevent dynamic change in protein degradation due to the aggregation of fluorescent molecules;

7. The fluorescent molecules used in the present invention exhibits a fast folding rate, thereby ensuring the timely, dynamic tracking of changes in protein stability.

The invention will be further illustrated with reference to the following specific examples. It is to be understood that these examples are only intended to illustrate the invention, but not to limit the scope of the invention. For the experimental methods in the following examples without particular conditions, they are performed under routine conditions, such as conditions described in Sambrook et al., Molecular Cloning: A Laboratory Manual, New York: Cold Spring Harbor Laboratory Press, 1989, or as instructed by the manufacturer. All the percentages or fractions refer to weight percentage and weight fraction, unless stated otherwise.

Unless otherwise defined, all the technical and scientific terms used in the present specification have the meanings as commonly understood by those skilled in the art. In addition, any method and material which are similar or equivalent with the contents disclosed herein can be applied in the present methods. The preferred methods and materials for carrying out the present invention described herein are only given as examples.

### EXAMPLE

Materials and Methods

CZ-1 Cell and CZ-1/R Cell

CZ-1 cell was from multiple myeloma cell line (i.e, BTZ drug-sensitive cell line); this cell line was in vitro intermittent-treated by small dose of BTZ, and subcultured for several passages, thereby obtaining BTZ resistance, marked as CZ-1/R (i.e., BTZ-resistant cell line).

CZ-1 cells can be obtained from Hou Jian Laboratory of Shanghai Changzheng Hospital (Hou J, Lin F Y, Zhang B, Zhang L Z, Ding S Q, Establishment and biological characteristics of human multiple myeloma cell line CZ-1. Chinese Medical Journal [2004, 117(1):115-119]); CZ-1/R cell lines was constructed by the inventor's Laboratories, and can be obtained from the inventor's laboratory (http://www.sibcb.ac.cn/PI.asp?id=129).

293 Cell

293 cell was obtained from ATCC (Maryland, USA).

Construction of Plasmid

Designed dual-fluorescent structures were assembled, and amplified by PCR reaction, and restriction enzyme sites were added, wherein two Flag labels were attached to C-end of mEGFP fragment, two Flag labels were attached to C-end of mRFP fragment, and Ub protein was Ub protein with K(R) saturation mutation, named as mEGFP_{fu}-mRFP_{f}. The three fragments were spliced, and inserted into vector pAG426GAL-ccdB-EGFP (Addgene) via Hind III and Xho I restriction sites, wherein EGFP structure was replaced by mEGFP-Ub-mRFP structure, thereby obtaining plasmid pAG426GAL-ccdB-mEGFP_{fu}-mRFP_{f}.

Primers for constructing plasmid pAG426GAL-ccdB-mEGFP_{fu}-mRFP_{f} are listed as follows:

HindIII-(a)-mGFPN1(5-3):
(SEQ ID NO: 5)
ACA aagctta atggtgagca agggcgagga
mGFPC1-FLAG2N1 (3-5):
(SEQ ID NO: 6)
tggacgagct gtacaag ggatctgactacaag
GWs3(5-3):
(SEQ ID NO: 7)
gggtctgactacaaggacgatgacgataagggcggagactacaaggacga
tgacgataagggctct
FLAG2C1-hUbN1(5-3):
(SEQ ID NO: 8)
acgatgacgataagggctct atgcagatctttgtgaGg
GWS5(5-3):
(SEQ ID NO: 9)
atgcagatctttgtgaGgaccctcactggcaGaaccatcacccttgaggt
cgagcccagtgacaccattgagaatgtcaGagccaGaattcaagacaGgg
GWS6(5-3):
(SEQ ID NO: 10)
gccaGaattcaagacaGggagggtatcccacctgaccagcagcgtctgat
atttgccggcaGacagctggaggatggccgcactctctcagactacaac
hUbK6RN3-mRFPN1 (3-5):
(SEQ ID NO: 11)
cactctctcagactacaacatccagaGagagtccaccctgcacctggtgt
tgcgcctccgcggtggtatggcctcctccgaggacgt
GWS8(5-3):
(SEQ ID NO: 12)
atggcctcct ccgaggacgt
GWS9(5-3):
(SEQ ID NO: 13)
ccgc cactccaccg gcgcc ggAtctgactacaaggacgat
GWS10(5-3):
(SEQ ID NO: 14)
acaaggacgatgacgataag TAG taactcgagtca

pAG426GAL-ccdB-mEGFP_{fu}-mRFP_{f} plasmid was digested by spe1 and XhoI restriction endonucleases, and blunted. PCDH-CMV-MCS-EF1 a-Puro vector (Addgene) was digested by Xba1, blunted, and then linked by T4 ligase, and sequenced as being correct. Plasmid PCDH-CMV-ccdB-mEGFP_{fu}-mRFP_{f} was obtained.

Mixing and Amplification of Human Gene Libraries

Human gene libraries were mixed by using Biomek FX automated workstation, and detected for titer. The original libraries were amplified for 100 times, and at 37° C. cultured for 18˜24 h. Clones were scraped from the plate, collected and well-mixed. And plasmids were massive-extracted.

LR Reaction and Identification of Human Gene Library

Human gene library is present in Entry clones containing attL sequence. LR reaction can occur directly between genes in the library and the inventor's target vector PCDH-CMV-ccdB-mEGFP_{fu}-mRFP_{f} (containing attR sequence) (technical details and all of operations can be found in Invitrogen GATEWAY™ Cloning Technology), to obtain human gene libraries with the presence of PCDH-CMV-ORFs-mEGFP_{fu}-mRFP_{f}. Clones were scraped, collected and well-mixed. And then plasmids were massive-extracted.

Construction of Dual-Fluorescent Cell Library

At 37° C. and 5% CO₂, 293 FT cell line was cultured in DMEM medium containing 10% fetal bovine serum, 10% NEAA and P/S antibiotics in an incubator. 293 FT cells were transfected with pCDH-CMV-ORFs-mEGFP_{fu}-mRFP_{f} library, wherein used helper plasmid was lentiviral expression system (LV100A-1, SBI), and the operation methods can be found in operating specification.

Construction of Dual-Fluorescent Control Cell Line

Plasmids pENTRY-RGS4, pENTRY-TP53, pENTRY-ARC, pENTRY-AXNA1, pENTRY-NFKBIB, and pENTRY-CDC25A were picked from hORFome library, and gateway reaction was conducted with target vector pCDH-CMV-ccdB-mEGFP_{fu}-mRFP_{f}, thereby obtaining target plasmids pCDH-CMV-RGS4-mEGFP_{fu}-mRFP_{f}, pCDH-CMV-P53-mEGFP_{fu}-mRFP_{f}, pCDH-CMV-ARC-mEGFP_{fu}-mRFP_{f}, pCDH-CMV-AXNA1-mEGFP_{fu}-mRFP_{f}, pCDH-CMV-NFKBIB-mEGFP_{fu}-mRFP_{f}, and pCDH-CMV-CDC25A-mEGFP_{fu}-mRFP_{f}, respectively. Viruses were packaged by using lentiviral expression system (LV100A-1, SBI). 293 FT cells were infected, and sorted by FACS, thereby obtaining dual-fluorescent overexpressing cell lines pCDH-CMV-RGS4-mEGFP_{fu}-mRFP_{f}, pCDH-CMV-TP53-mEGFP_{fu}-mRFP_{f}, pCDH-CMV-ARC- mEGFP_{fu}-mRFP_{f}, pCDH-CMV-AXNA1-mEGFP_{fu}-mRFP_{f}, pCDH-CMV-NFKBIB-mEGFP_{fu}-mRFP_{f}, and pCDH-CMV-CDC25A-mEGFP_{fu}-mRFP_{f}, respectively.

Dual-fluorescent overexpressing cell lines pCDH-CMV-RGS4-mEGFP_{fu}-mRFP_{f}, pCDH-CMV-TP53-mEGFP_{fu}-mRFP_{f}, pCDH-CMV-ARC-mEGFP_{fu}-mRFP_{f}, pCDH-CMV-AXNA1-mEGFP_{fu}-mRFP_{f}, pCDH-CMV-NFKBIB-mEGFP_{fu}-mRFP_{f}, pCDH-CMV-CDC25A-mEGFP_{fu}-mRFP_{f} were treated by Bortezomib (final concentration of 1 μM) for 6 hrs, with DMSO as the control. Changes in EGFP/RFP ratio were detected by FACS. And then cells were lysed, and changes in the protein abundance were detected by Western assay.

8-Section Sort of Dual-Fluorescent Cell Library

Cells in the dual-fluorescence cell library containing Human ORFeome V5.1 genes were digested, blown as single cell, resuspended in a medium, filtered through 40 μm filter, and sorted by FACS AriaII cell sorting system (BD company). When cells were sorted, cell population with high RFP signal was firstly selected, and then the selected cells were sorted into 8 sections based on fluorescence ratio of mEGFP/mRFP.

Extraction and Microarray Hybridization of Genomic DNA of Cells in Sections

At 55° C., cells in each section were lysed with a lysis buffer for 16 hrs, the composition of which is 10 mM Tris-HCl (8.0), 10 mM EDTA, 0.5% SDS, 0.2 mg /ml proteinase K and 25 μg/ml RNase A. And then 0.2 M sodium chloride was added. Genomic DNA was extracted by phenol-chloroform, purified by chloroform, precipitated by ethanol, and then dissolved in 10 mM Tris-HCl (8.0) and 0.1 mM EDTA buffer. For quantitative amplification of ORF-mEGFP_{fu}-mRFP_{f} structure integrated in the genome, PCR amplification was conducted by using genomic DNA as a template, wherein PCR primers are listed as follows:

Forward primer:
(SEQ ID NO: 15)
CCTGGAGACGCCATCCACGCTG
Reverse primer:
(SEQ ID NO: 16)
TAATACGACTCACTATAGGGAGCTCCTCGCCCTTGCTCACCATTAAGCT.

T7 RNA polymerase promoter site was contained in the reverse primer. Amplification was conducted by using EX-Taq enzyme (Takara). And then, using PCR product as a template, in vitro RNA amplification was performed using T7 MEGAscript (Ambion) kit, the amplified RNA was purified using RNeasy mini kit, and the purified RNA was labeled with Cy3 and Cy5 (Cat #PA13105, GE Healthcare Bioscience, Pittsburgh, Pa., US), and then purified using RNeasy mini Kit (Cat #74106, QIAGEN, GmBH, Germany). Specific details can be found in operation specification.

Chip Hybridization

Then, chip hybridization was performed based on the amount (Cat #5188-5242, Agilent technologies, Santa Clara, Calif., US) in Hybridization Oven (Cat #G 2545A, Agilent technologies, Santa Clara, Calif., US). After 17 hours, the chip was washed (Cat #121, Thermo Shandon, Waltham, Mass., US) (Cat #5188-5327, Agilent technologies, Santa Clara, Calif., US), fixed, and dried (Cat # 121, Thermo Shandon, Waltham, Mass., US) with Gene Expression Wash Buffer Kit (Cat #5188-5327, Agilent technologies, Santa Clara, Calif., US). All of operations can refer to operation specification. The same probe signals were combined and averaged. For each ORF, the sum of signal values in 8 sections was normalized to 1, and then PSI and STDEV values were calculated. Specific details for the calculation can be found in literatures (Hsueh-Chi Sherry Yen, et al., 2008, science).

Search for Important in vivo Biological Pathways and Key Proteins, the Stability of which is Affected upon Bortezomib Treatment

For finding Bortezomib-sensitive protein, ORFs-mEGFP_{fu}-mRFP_{f} cell library is divided into two groups: for control group, DMSO was added; and for experimental group, Bortezomib was added (1 μM final concentration). And then cells were sorted by FACS in 8 sections, and the effects of Bortezomib treatment on the stability of genome-wide proteins were detected.

Data were deeply analyzed by system biology.

GO Annotation and Analysis

For each ORF, change in PSI was calculated by the inventors under DMSO and Bortezomib treatment conditions. ORFs were ranked according to ΔPSI/PSI by the inventors, and then GO enrichment analysis was applied to the top 15% of genes in the rank. All of the probes on the chip were used as control genes, and DAVID online software was used for GO enrichment analysis (Glynn Dennis Jr, 2003; da Wei Huang, 2009) by the inventors.

Signaling Pathway Enrichment Analysis

DAVID online software was used to search 14 functional annotation databases, such as Kyoto Encyclopedia of Genes and Genomes (KEGG) and GO annotation, Biocarta pathway, BBID, and PANTHER pathway, for signaling pathways, the significance of which is changed.

Protein-Protein Interaction Network

For better understanding overall effects on cell signaling networks upon the addition of Bortezomib, protein-protein interaction network analysis was applied to the top 5% of genes in ΔPSI/PSI rank selected by the inventors, and online tool STRING database (Snel B, 2000) was used.

Small Compound-Protein Interaction Analysis (CPI)

Protein-small compound interaction network is important for understanding the function of molecules and cells. Compound-protein interaction network was analyzed using STITCH (‘search tool for interactions of chemicals’) (Kuhn et al., 2008) (2.0 version) (http://stitch2.embl.de/) online database.

MTT Detection of Cell Viability

The concentration of the cell suspension was adjusted, the cells were plated at a density of 1000-10000 cells to be tested/well, a concentration gradient of Bortezomib, 17-AAG (Selleck Chemicals) or C75 (Cayman Chemical) was added, 4-6 replicates for each concentration. Plates were incubated for 24-72 hours. To each well was added 20 ul MTT (3-(4,5-dimethyl-2-thiazole)-2,5-diphenyl tetrazolium bromide) solution, culture was continued for another 4 hrs. To each well was added 150 ul of dimethyl sulfoxide, shaken on a shaker at low speed for 10 mins for thoroughly dissolving the crystals. Absorbance of each well was measured on an enzyme-linked immune detector at OD 490 nm (570 nm).

Interaction Parameter CI (Combination Index) Analysis

CompuSyn software (http://www.combosyn.com/) was used to analyze BTZ and 17-AAG or C75 interaction mode. Chou-Talalay principle was applied to interaction equation (Chou T C, P Talalay, Quantitative Analysis of dose-Effect relationships: the Combined Effects of Multiple Drugs or Enzyme inhibitors. Advances in Enzyme Regulation 02/1984; 22: 27-55), that is, when CI=1, >1, or <1, the drug can be deemed as exhibiting additional, antagonistic or synergistic effect, respectively. CZ-1 cells were added into 96-well plate at 5,000 cells per well, and corresponding concentrations of BTZ were added respectively in combination with 17-AAG or C75, and after 24 hr, cell viability was detected using MTT.

### Example 1

### Efficient Detection of Relative Stability of a Protein by Dual-Fluorescent Protein System

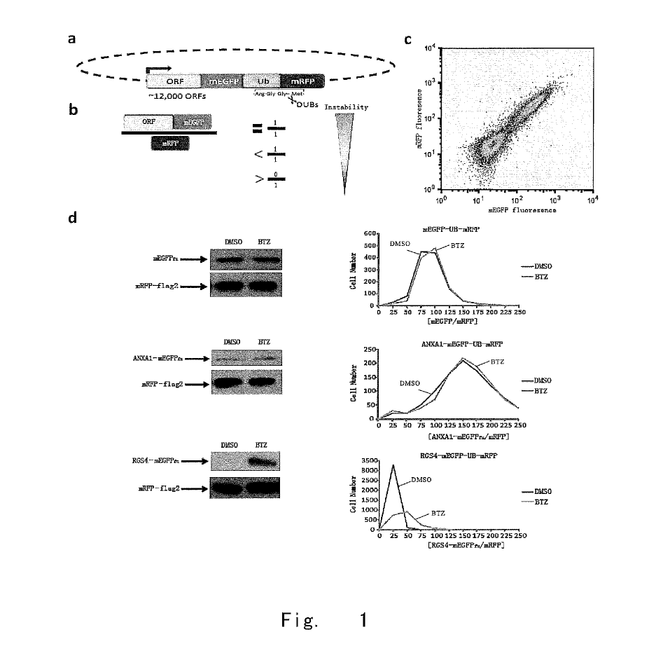

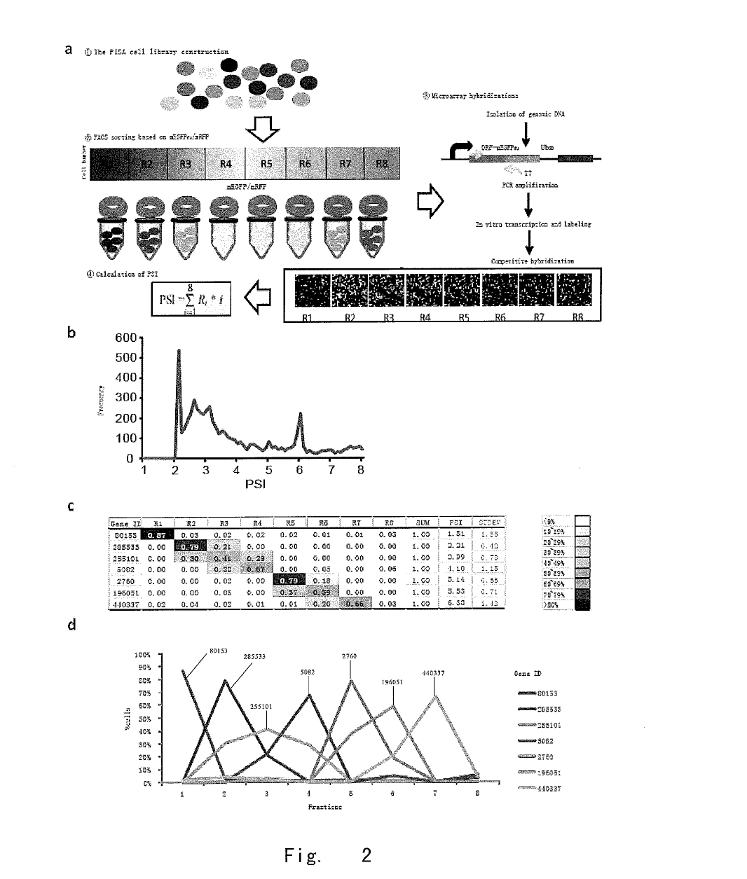

A dual-fluorescent protein structure was designed by the inventors, wherein mEGFP was fused to C-terminal of target protein, intermediate linker protein is ubiquitin with K(R) saturated mutation, and internal reference fluorescent protein is mRFP, thereby forming a structure of mEGFP_{fu}-mRFP_{f}, and wherein mEGFP is of the sequence of SEQ ID NOs: 1 and 2, mRFP is of the sequence of SEQ ID NO: 3, 4, Ub_{K0} is of the sequence of SEQ ID NOs: 17 and 18. A schematic view of the structure is shown in FIG. 1a, the principle for the relative quantification of target proteins is shown in FIG 1b. Artificially designed recombinant dual-fluorescent protein structure was constructed on an expression plasmid of mammalian cell by the inventors, and constitutively expressed by using CMV promoter.

mEGFP_{fu}-mRFP_{f} without any exogenous gene being fused was constructed into an eukaryotic expression vector, and 293 FT cells were infected through lentivirus, thereby successfully constructing a stably transfected cell line with mEGFP_{fu}-mRFP_{f} being fused. Through FACS, mEGFP-UB_{K0} was detected as having the same stability as mRFP, both of which are very stable. See FIG. 1C, wherein cells in low signal area are negative control cells.

For detecting the relative stability of a gene, gene ARC, TP53, CDC 25A were fused to N-terminal of mEGFP in mEGFP-UB_{K0}-mRFP structure through gateway recombinant technology. 293 FT cells were infected through lentivirus, thereby successfully constructing a stably transfected cell line with ARC-mEGFP_{fu}-mRFP_{f}, TP53-mEGFP_{fu}-mRFP_{f}, CDC25A-mEGFP_{fu}-mRFP_{f} being fused. Through FACS, the inventors found that different genes correspond to different protein stabilities. See FIG. 5.

For verifying whether the dual-fluorescent protein will be cleaved by deubiquitinating enzyme, the inventors tested the protein expressed by TP53-mEGFP_{fu}-mRFP_{f} vector as an example. The original band of the fusion peptide which has just been translated, and two small bands (TP53-mEGFP fusion protein band and mRFP band) formed through cleavage with ubiquitination enzyme were verified through western blot (see FIG. 7). It demonstrates that the fusion polypeptide provided in the present invention was successfully cleaved by ubiquitination enzymes, and two protein fragments are formed as expected.

For testing whether the dual-fluorescent protein structure can monitor the effects of exogenous stimulus or disturbance on protein stability in real-time, AXNA1-mEGFP_{fu}-mRFP_{f} and RGS4-mEGFP_{fu}-mRFP_{f} cell lines were constructed by the inventors, and the response of the cell line toward anti-cancer drug Bortezomib was detected. It is demonstrated that changes in protein stability can be sensitively and specifically detected by the dual-fluorescent system of the present invention, based on FACS and western blotting detection results. See FIG. 1d.

### Example 2

### Genome-Wide Detection of Protein Stability by mEGFP-UB_{K0}-mRFP Dual-Fluorescent Protein System

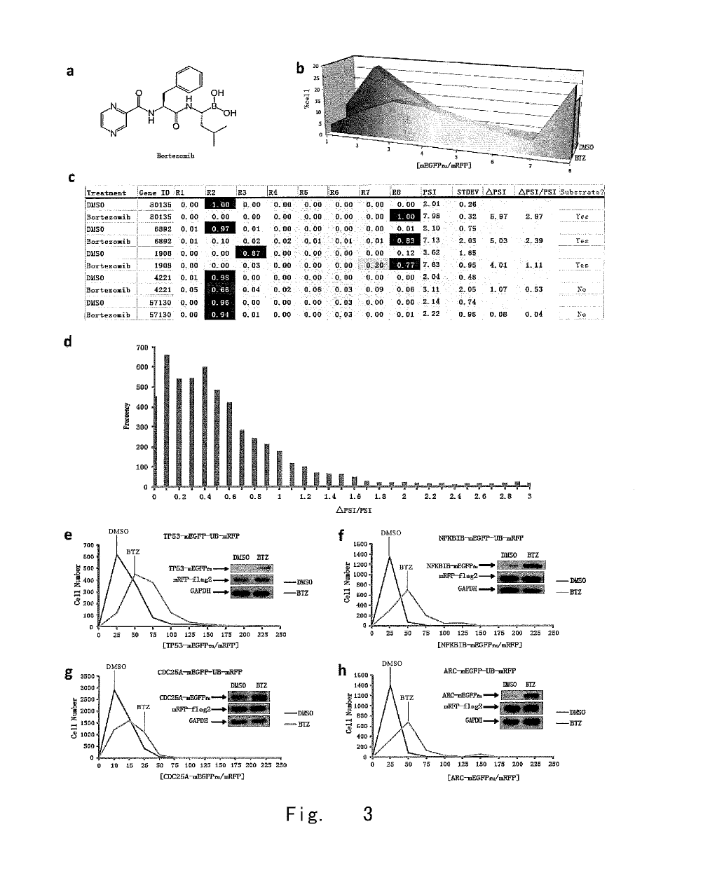

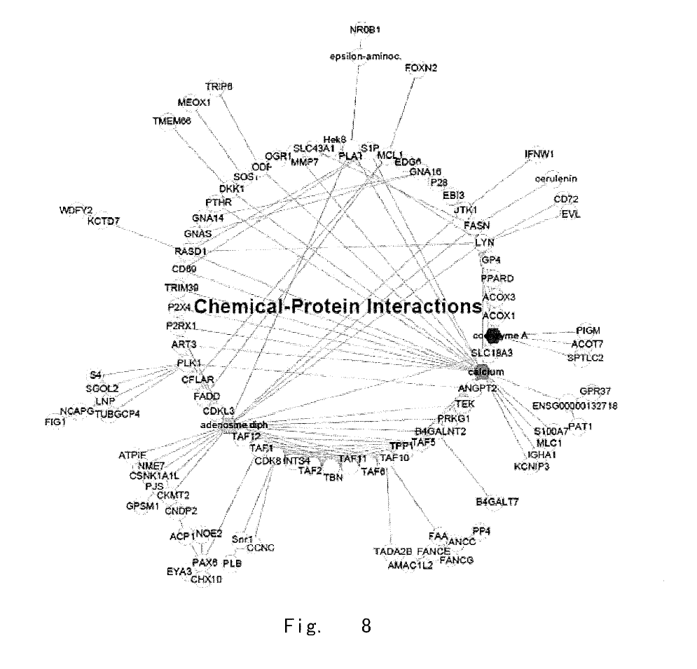

C-termini of about 15000 ORFs were fused to mEGFP-UB_{K0}-mRFP structure through gateway recombinant technology by using human ORFeome V5.1 as target gene library. 293 cells were infected through lentivirus infection by using a gene library with mEGFP_{fu}-mRFP_{f} being fused, thereby constructing a dual-fluorescent cell line with library genes being integrated. See FIG. 6. And then the library was sorted into 8 sections based on the ratio of mEGFP/mRFP by FACS technology. Genomic DNA from cells in each section was extracted, in vitro cRNA was amplified with PCR products as templates, and then the product was subjected to chip hybridization. Data were processed by using Composite loess normalization program in Bioconductor package limma, and relative stability parameter PSI (Protein Stability Index) for each gene was calculated. The flow chart can be found in FIG. 2a. The distribution of relative stability parameter of protein can be found in FIG. 2b, and representative data can be found in FIG. 2c, d. FIG. 2d is a broken line graph plotted based on the data shown in FIG. 2c, wherein responsive genes are distributed as single peaks.

### Example 3

### Detection of Candidate Genes Responsive to Anti-Cancer Drug Bortezomib at Protein Stability Level by Dual-Fluorescent Cell Library

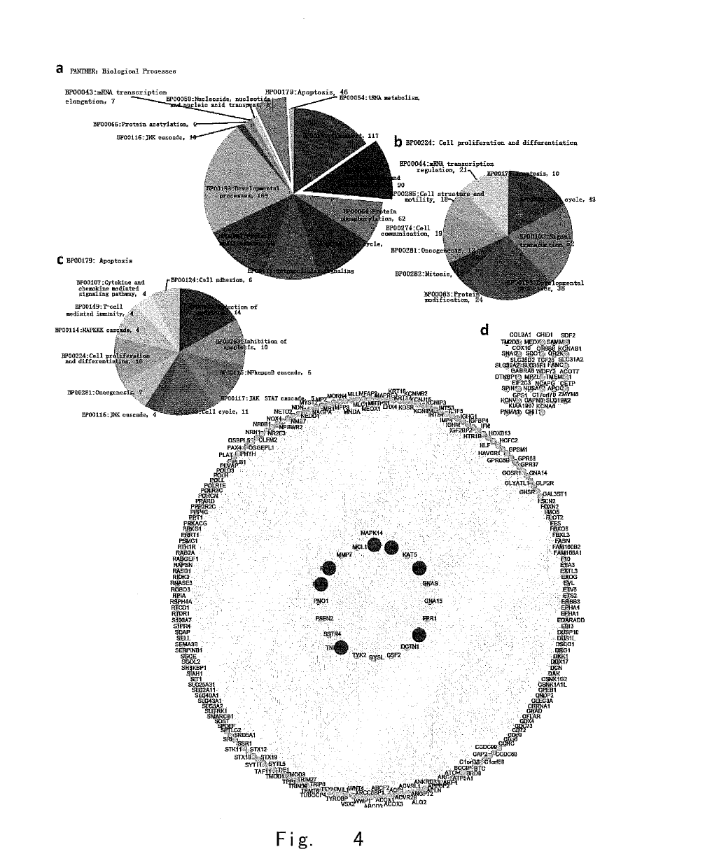

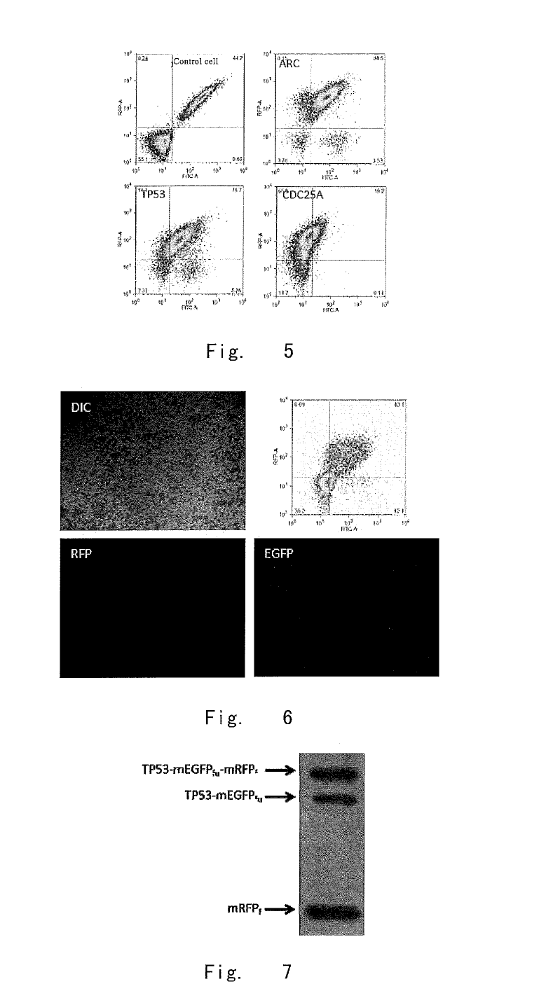

DMSO (control treatment) and Bortezomib (experimental treatment) were used to treat cells in dual-fluorescent cell library containing human ORFeome V5.1 gene library, respectively, by the inventors. And then according to the scheme shown in FIG. 2a, PSI values for each library gene under DMSO and Bortezomib were calculated, ΔPSI=PSI_{BTZ}−PSI_{DMSO} was obtained, all of the genes were ranked based on ΔPSI and significant genes were analyzed through bioinformatics. FIG. 3a shows the chemical structure of anticancer drug Bortezomib; FIG. 3c, d are representative data and substrate-responsive proteins; and FIG. 4 is bioinformatics analysis results.

### Example 4

### Guide to Rational Drug Combination from CPI Analysis of ProTA Data

Protein-small compound interaction network is important for understanding the function of molecules and cells. Therefore, the inventors have studied small compounds significantly interacting with BTZ effectors using STITCH (search tool for interactions of chemicals) online database for exploring cellular mechanisms between the cytotoxicity of Bortezomib and the development of drug resistance. DMSO (control treatment) and Bortezomib (experimental treatment) were used to treat cells in the library obtained in Example 2, respectively. And then according to the scheme shown in FIG. 2a, PSI values for each library gene under DMSO and Bortezomib were calculated, ΔPSI=PSI_{BTZ}−PSI_{DMSO} was obtained, all of the genes were ranked based on ΔPSI and CPI (compound-protein interaction) analysis was applied to the top 250 hits. CPI analysis results are shown in FIG. 8. From CPI analysis results, it is demonstrated that small compounds adenosine diph (replaced by analog 17-AAG) and coenzyme A (using FASN inhibitor C75) were specifically enriched by the inventors.

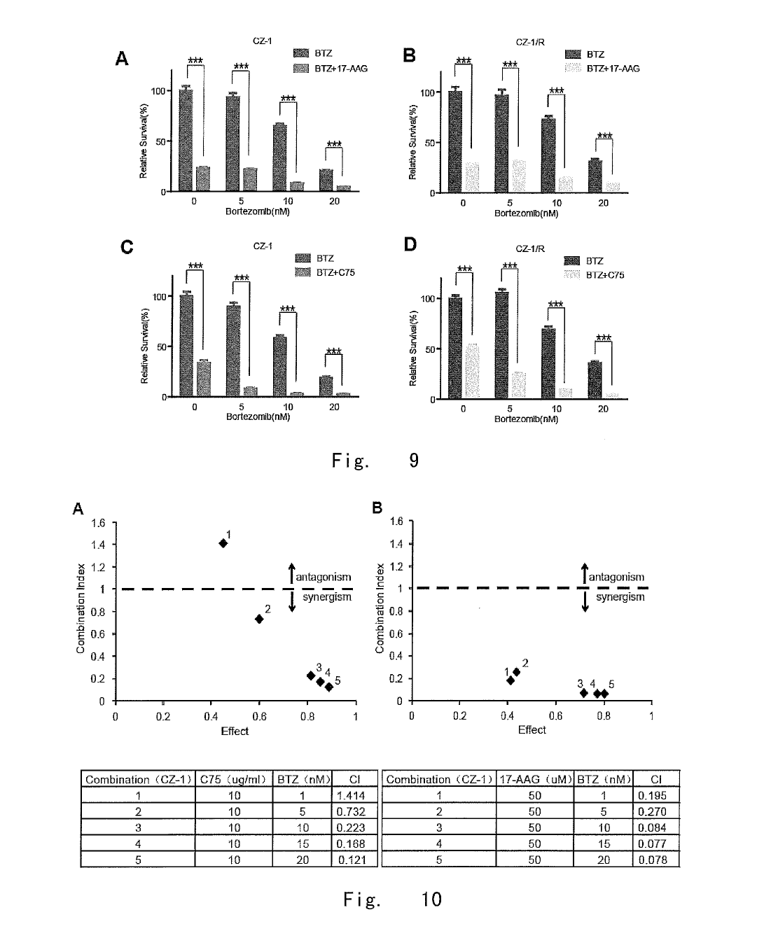

Subsequently, the inventors have verified the effects of BTZ in combination with 17-AAG or C75 by using CZ-1 and CZ-1/R cell line as verification cell line, and interaction parameter CI (Combination index) was calculated, that is, interaction mode of drug BTZ with 17-AAG or C75 was analyzed, and the results are shown in FIGS. 9 and 10. FIG. 9, showing the effects of BTZ in combination with 17-AAG or C75 detected by MTT, shows that BTZ in combination with 17-AAG or C75 exhibits significant killing effects on BTZ sensitive cell line CZ-1 or BTZ-resistance CZ-1/R cell line. FIG. 10 shows that BTZ at concentration of 5, 10, 15 and 20 nM exhibits synergistic effects with 17-AAG, and BTZ at all of the tested concentrations exhibits synergistic effects with C75.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A genetic construct with a structure of the following formula:
5′-A+B+C+D+E-3′,
wherein,
A represents a promoter;
B represents a coding sequence for a fusion protein consisting of a target protein and a first marker protein;
C represents a coding sequence for a linker peptide;
D represents a coding sequence for a second marker protein; and
E represents a terminator;
wherein B and D can be interchanged.

2. The genetic construct according to claim 1, wherein B, from 5′ to 3′, comprises a coding sequence for the target protein and a coding sequence for the first marker protein.

3. The genetic construct according to claim 1, wherein the linker peptide is selected from: ubiquitin, truncated ubiquitin, ubiquitin mutants and ubiquitin-like protein, 2A peptides and the like; preferably, ubiquitin; most preferably, ubiquitin with K(R) saturation mutation.

4. The genetic construct according to claim 1, wherein the first marker protein and the second marker protein are fluorescent proteins.

5. The genetic construct according to claim 1, wherein the coding sequence of the target protein comprises all of the genes in human genomic library human ORFeome V5.1.

6. A polypeptide with the structure of a following formula:
B1+C1+D1,
wherein,
B1 represents a fusion protein consisting of a target protein and a first marker protein;
C1 represents a linker peptide; and
D1 represents a second marker protein;
wherein B1 and D1 can be interchanged.

7. A vector comprising the genetic construct according to claim 1.

8. A cell comprising the genetic construct according to claim 1.

9. A cell library consisting of the cells according to claim 8.

10. A method for detecting the stability of one or more target proteins, comprising the following steps:
(1) constructing the cell library according to claim 9;
(2) culturing the cell library obtained in (1) under the specific conditions and control conditions;
(3) determining the ratio of the first marker protein to the second marker protein in the cell library cultured under the specific conditions and control conditions; and
(4) drawing a conclusion that the stability of the target protein is changed or not, based on the ratio of the first marker protein to the second marker protein in the cell line cultured under the specific conditions and control conditions; or:
based on the ratio of the first marker protein to the second marker protein, separating the cell into n sections (n≧2) through flow cytometry, detecting the expression of each target gene in each section by DNA chip technology to calculate the distribution of the target genes in each section, and comparing the distributions of target genes in cell lines cultured under the specific conditions and control conditions, thereby drawing a conclusion that the stability of the protein encoded by the target gene is changed or not.

11. A cell comprising the genetic construct according to the vector according to claim 7.
